(19) United States
(12) Patent Application Publication
(10) Pub. No.: US 2003/0135094 A1
(43) Pub. Date: Jul. 17, 2003

(54) Apparatus for remote medical examination

(76) Inventors: Miklos Illyes, Budapest (HU); Jozsef Beres, Budapest (HU)

Correspondence Address: WILLIAM COLLARD, COLLARD & ROE, P.C., 1077 NORTHERN BOULEVARD, ROSLYN, NY, 11576, US

(21) Appl. No.: 10/297,273

(22) PCT Filed: Jun. 11, 2001
(86) PCT No.: PCT/HU01/00068

(30) Foreign Application Priority Data
Jun. 15, 2000 (HU) P 0002273

(51) Int. Cl. A61B 5/00

(52) U.S. Cl. 600/300

(57) ABSTRACT

A system of apparatuses (1) for examination of status and readiness for cooperation (compliance) of patients under treatment and/or for preparing their therapy, using remote data processing, the said system comprises patient's units (3) at the persons being under observation and/or treatment and at least one unit installed at the physician and/or in a health center. The characteristic feature of the system according to present invention is that the patient's units (3) are equipped with: a) an input connection, which is compatible with the output of an instrument (5) measuring one or more physiological data of the patient; b) a signal handling unit (32), which filters from noise, amplifies and/or digitizes the incoming measured data according to necessity; c) a control unit (38) equipped with a program unit (33), a data storage unit (34), a background database interface unit (35), a real time unit (31) and a display unit, issuing preferably a warning light or sound signal indicating the points of time to perform the actual examinations (36), which controls the said units; d) a program unit (33) connected to the said control unit (38) and provided with a program, which controls the measurement of physiological data performed by the measuring instrument (5) among other functions; e) a data storage unit (34) connected to the said control unit (38), which contains algorithms to process and evaluate the measured data and stores the measured data; and f) a background database interface unit (35) connected with the said control unit (38), which is applicable to establish connection with the background database (2) of the system of apparatuses to transfer data to or receive them from the said database. Furthermore the physician's units (4) are equipped with: an interface unit (44) designed for I/O operations to send immediately data to or receive them from a determined group of patient's units (3) and perform the same transfers with the background database (2) of the system. Finally, the system of apparatuses (1) comprises one or more background databases (2), which are equipped with an interface unit (25) designed for I/O operations, which establishes and maintains simultaneous connections functioning in time-sharing mode with the patient's units (3) and the physician's units (4).

### THE FIELD OF INVENTION

The subject matter of this invention is a system of devices which serves to examine status and readiness to cooperation (compliance) of persons being under medical treatment and/or to prepare their therapy by means of teleprocessing, the said system comprises several patient's unit installed at the observed and/or treated persons and at least one unit installed at the physician and/or in the health center.

In the field of the difficulties of health protection systems can be shown in the example of hypertensive vascular diseases. After the contagious diseases have been considerably repelled, the cardiovascular diseases moved into foreground. These cause of death leads the statistics of morbidity and mortality, e.g. their proportion is 51% in Hungary. In the background of this group of diseases the presence of hypertension appears in a determining proportion either as cause or as concomitant disease defining the progression of course of diseases. The diagnosis and appropriate treatment should become a highly preferred target in the medical practice. Actually this disease without symptoms is diagnosed by single occasional measurements of blood pressure (see FIG. 1). The patient receives a therapy with or without medicaments or combination thereof, he is treated either in a hospital or at an outpatient service, according to his status and on the base of results of complementary examinations. The blood pressure of the patient is controlled thereafter usually by regular measurements (performed always in the consulting room). The efficacy of diagnose and treatment of hypertension based on occasional measurements is, however, very low. Over and above the fact that about 20 to 30% of patients, having normal blood pressure, are diagnosed and treated erroneously as hypertensive because of the so-called “white cloak effect” (the psychological stress caused by the physician and atmosphere of the consulting room increases the blood pressure of some patients), even the proportion of adequately treated patients (RR<140/90 millimeter of mercury column) is disappointingly low in the system of treatment based on the occasional measurements, it attains only 20 to 27% according to Hungarian and international inquiries. As consequence of these facts unnecessary expenses occur for medicaments on one hand, on the other hand we did not manage to reduce the frequency of main complications caused by hypertension (like apoplexy, heart infarct, heart insufficiency) as required. By this way the treatment of patients suffering from hypertension has become one of main encumbrances of health protection of present day both in professional and in financial sense.

### THE STATE OF THE ART

It is one of the main causes of nationwide inefficiency of treatment against hypertension, that the diagnosis and treatment is based even now on the occasional measurements in the public health clinics. Another cause of similar importance is that since this disease remains without symptoms during several years, so the motivation of patients reduces by the time to take medicaments prescribed by physician and to take care of this disease, and it is proven, that only 40% of patients take the medicament according to physician's prescriptions after a year. The physician sees that the treatment has no results, and he is in a troublesome situation, because he cannot find out, if the high blood pressure is caused by the improper medicament chosen, the progress of disease or simply because the patient does not take the prescribed medicament in. Physicians can base their decisions only onto their insight into human nature. Since only 20 to 27% of all treated patients receive optimal therapy, the frequency of complications is extraordinarily high. The quality of life changes for the worse for a number of patients, the complications cause early death in numerous cases.

The diagnostic procedures, which provide better and more accurate diagnosis and control more reliable the successful treatment, become more and more widespread. The All-day long Blood Pressure Monitoring system (ABPM) excels among them, which follows the daily profile of blood pressure under the usual activity of patient, using a non-invasive method. However its usage is spreading, this method of examination is utilized only by a small group of specialists. This fact is based on the high price of equipment on one hand and on the shortage of special knowledge required for the usage of said method, on the other hand.

It is known a medical inspection system according to the U.S. Pat. No. 5,544,649 description. The system consists of a central station and a series of patient's local stations arranged around it, and also a physician's office may be connected to it. Each patient unit is provided with measuring instruments necessary for the continuous examination of the patient's status. The results appear on the displays being in the central station, from where they may control the patient's instruments by remote control. Practitioners do the inspection, and the patient and his (her) practitioner may continuously see one the other by TV monitors. The physicians working at background have also TV monitor and phone, thus they also can see the patients, and may give advices. The base of the solution of the U.S. Pat. No. 5,911,687 is also an information network, composing an “electronic clinic”. There is a central computer, which comprises the list of physicians, arranged according to their medical activity and access address. A similar register is introduced about the pharmacies. A searching application searches the suitable physician and contacts his or her terminal with the patient's one as answer to the asks arrived from the patient's terminals. On the base of the said register the central computer recognizes the returning patients, connects him or her to the physician who made previously the treatment, and the said physician receives the documents of the patient. No one of those examples gives solution to the problems of the district doctors (i.e. home-doctors), not of help their work to make it easier.

In the practitioners inspection service according to the U.S. Pat. No. 5,544,649 the patient's terminals have no instrument for automatic controlling and notification of the time of examination and to take the medicament in. The central station is not an automatic co-ordinating unit with an electronic medical-science background library, but a place for the practitioners for manual remote-controlling the patient's instruments and watches them. On the first place this is a visual inspection system. The computer centre controls the data-transferring processes, but it makes neither medical evaluation, nor reports to the district doctors. Despite of the presence of the physicians and the possibility to advise, there are no patient-physician contacts, only patient-practitioner. This system has no means to observe and evaluate the “compliance” of the patients. The other solution, the doctor-searching system according to the U.S. Pat. No. 5,911,687, suitable neither the continuous home examination of the patient, nor to develop an up to date computer-assisted district doctor network. The contact between the patient and the physician remains occasional even for a returning patient and the necessary instruments are not present to inspect if the prescribed therapy is performed.

### THE DISCLOSURE

The aim of this invention is to develop a system and a conjoint group of apparatuses, which complies with the requirements arisen in line with the present level of technology and wishes of medicine.

In connection with the FIG. 1 it is obvious the advantage of the proposed treatment under home control. The FIG. 1 shows the modernization of the treatment of hypertension, comparing of the actually existing treatment with the proposed one. In the FIG. 1 used indications are summarized below.

A. The Actually Existing Treatment

A-1 Case (,,causal”) controll,, ABPM

A-2 Patients found as hypertensitive

A-3 TREATMENT: Ambulant or Clinical or Altogether (With or without medicament)

Results

20-25% of patients found as pseudo-positive (because of “white cloak effect”)

at most 30% of patients receives adequate treatment

the rest part of patients are improperly treated

A-4 After a year 40% of treated patients does not take the medicaments

A-5 COMPLICATION!

A-6 AGGRAVATION OF DISEAS Apoplexy (stroke), infarct, and other consequences

A-7 DECEASES 51% of all decease are caused by the hypertension

B. The Proposed Treatment

B-1 Case (,,causal”) controll,, ABPM

B-2 Patients found as hypertensitive

B-3 TREATMENT UNDER HOME CONTROL and MEASUREMENT

Results

Round the clock control and treatment

,,white cloak effect” is eliminated

the series of measured data increases

number of improperly treated decreases better diagnosis and therapy; more than 30% are the properly treated

B-4 The co-operation between physician and patient (compliance) improves as result of continuous control and instructions

B-5 THE COMPLICATIONS ARE REDUCED

B-6 REDUCTION of number of patients to be treated and the time required for treatment

B-7 REDUCTION of decease, the number of disabled person requiring provision and provision costs.

The invention is based on the recognition that in line with social development the number of people entitled to health service grows, but the network of medical institutions cannot be developed according to growing demand, because the part of public expenses to be allocated to health assurance either cannot be increased or they can within very strict limits. As consequence of this situation the load of public medical clinics and other institutions of out-patient service grows, the time used for examination of a patient reduces, this way the quality of service also reduces, but more and more people come upon clinical treatment. The load of social insurance also grows. The targets are the following: the possible early recognition of disease, to provide the patients with proper diagnosis and therapy, to assure prevention and to increase capacity of attending physicians together with reduction of their workload. These targets can be ideally accomplished if all the procedures suitable for automation are automated, but everything is entrusted to the physician which requires essentially the physician's individual ability of consideration, recognition and decision-making. An extraordinarily wide variety of possibilities is offered by communication equipment provided by the information society to establish the ideal solution, the extent of utilization thereof should be grown for medical purposes. We consider, that in the era of “evidence based medicine” the data processing and evaluation can be automated by means of high-level algorithms, the results thereof provide the attending physician with information and preparation at a level of a clinical conference.

The invention solves the problem by an inventive idea that it includes a data processing and evaluating center with high intelligence between patient and physician, which center is “invisible” for the patient, i.e. it does not cover the physician, and it provides the physician with complete service at the same time without to separate him from the patient. According to inventive idea the data processing and evaluating center should not be the same for all diseases, the task of service will be in fact forwarded to one of a series of databases having specialized information according to the character of disease recognized. All these databases appear as a sole server to the physician, either the center sends the regular report or the physician calls it asking for extraordinary information. The decentralization of such type of the data processing and evaluating center enables to include specialized new knowledge into system to maintain and develop it effectively.

The objective can be accomplished by means of a specific group of medical measuring instruments and electronic data communication equipment wherein the said devices are distributed between the patient, the physician and the data processing and evaluating center by the most expedient way providing on-line connections between them at the same time.

The invention uses telecommunication methods being known in it, like telephone, radio and the modern connection methods of Internet or e-mail. The information transmission lines constitute a separate network of virtual connection according to inventive idea, which network establishes a connection between the patient, physician and evaluating center for the distribution of specific tasks.

The inventive solution complying with the target is a system of apparatuses to monitor status and readiness for cooperation (compliance) of patients under treatment and/or to prepare their therapy, the said system comprises patient's units installed at the patients, at least one unit installed at the physician and at least one unit installed in the medical center. The system according to invention has the following characteristic features:

The patient unit is equipped with

An input connection which is compatible with the output one of one or more measuring instruments which measure one or more physiological data of patient and issue the measured data using electric signals;

A signal handling unit which filters noise, amplifies and/or digitizes the signals according to necessity;

A control unit, which comprises a connecting program unit, a data storage unit, an interface unit to the background database, a real time control unit attached to said controller and preferably a display unit, which issues light and/or sound signals notifying the points of time to perform measurements;

A programming unit attached to said control unit which is provided with a program and controls the work of instruments measuring the physiological data;

A data storage unit attached to said control unit which contains points of time when the measurement should be performed, the algorithms to process and evaluate the measured data and the measured data themselves;

An interface unit to the background data base attached to said control unit, which is applicable to establish connection to one or more background databases of said system, to send data thereto or receive data therefrom;

Furthermore the physician's unit is equipped with

An interface unit designed to perform I/O operations either to send or receive data to or from one or more background databases or to send or receive data to or from a defined group of patient's units through an immediate connection;

Finally, the system comprises one or more background databases; each of them is equipped with

An interface unit designed to perform I/O operations and maintaining connections in time-sharing mode with the patient's units and with physician's ones.

The system according to invention can be advantageously characterized by that the physician's unit is equipped with an operative unit, which is provided with a system of programs applicable to change the plans of therapy and examinations of patient's units belonging to it.

The system according to invention can be also advantageously characterized by, that the background database is equipped with a coordination unit, which transmits information to patient's units and to physician's units and alarm signal to ambulance units of quick intervention in any emergency case and with a data processing and evaluating unit, connected to said coordination unit, which evaluating unit processes and evaluates information incoming from the patient's units, draws up medical report for physicians and archives data in a background storage unit for later usage.

Furthermore, the system according to invention can be advantageously characterized by, that the background database is equipped with a background storage unit, which contains also data and algorithms relating to diagnosis and therapy in the designed special field of medicine and with a handling unit, connected to said data processing and evaluating unit.

The system according to invention can be advantageously characterized by, that the background database comprises a database, containing data, identifying the patients and characterizing their status, and a patients' database, containing data of patients pertaining to a physician, as well as a physician's database, both connected to said coordination unit and controlled thereby.

The system according to invention can be also advantageously characterized by, that the background storage unit is also designed to keep a journal of events, arisen in the system, measures being met and other data.

A preferred embodiment of the system according to invention can be characterized by that the background database, preferably its background storage unit contains evaluating algorithms of mathematical statistics applied to medicine.

Another preferred embodiment of the system according to invention can be characterized by, that the physician's unit is equipped with a program storage unit, which is provided with a system of programs applicable to perform searches and queries in one or more background data bases of the system.

The physician's unit of said system according to invention can be also advantageously characterized by, that it contains plans of examination and therapy of patient's units pertaining to it, the data of patients and those of recent notices (reports) of measurements of required number.

Finally, the system according to invention can be characterized by, that results of examinations and their processed data cannot be changed or deleted by physician's unit.

The system according to invention is presented by embodiments on the base of drawings attached without limitation of either the applicability of said system or the scope of protection claimed to the embodiments presented.

### DRAWINGS

FIG. 1: A draft flowchart of treatment of patients suffering from hypertension in the existing system and in the proposed one.

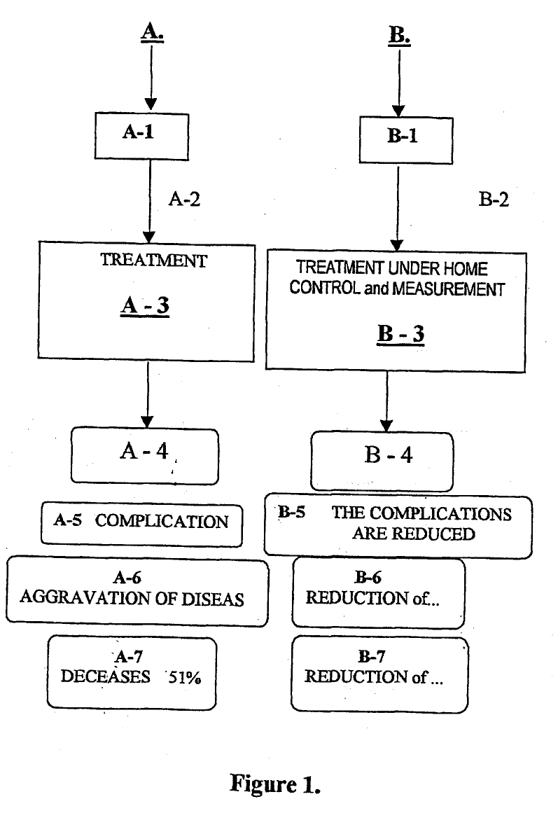

FIG. 2: A block-scheme of the system of apparatus according to invention.

FIG. 3: A flow-chart of the work of system of apparatuses according to invention.

FIG. 4: A simplified flow-chart of the work of patient's unit according to invention.

FIG. 5: A simplified flow-chart of the work of background database according to invention.

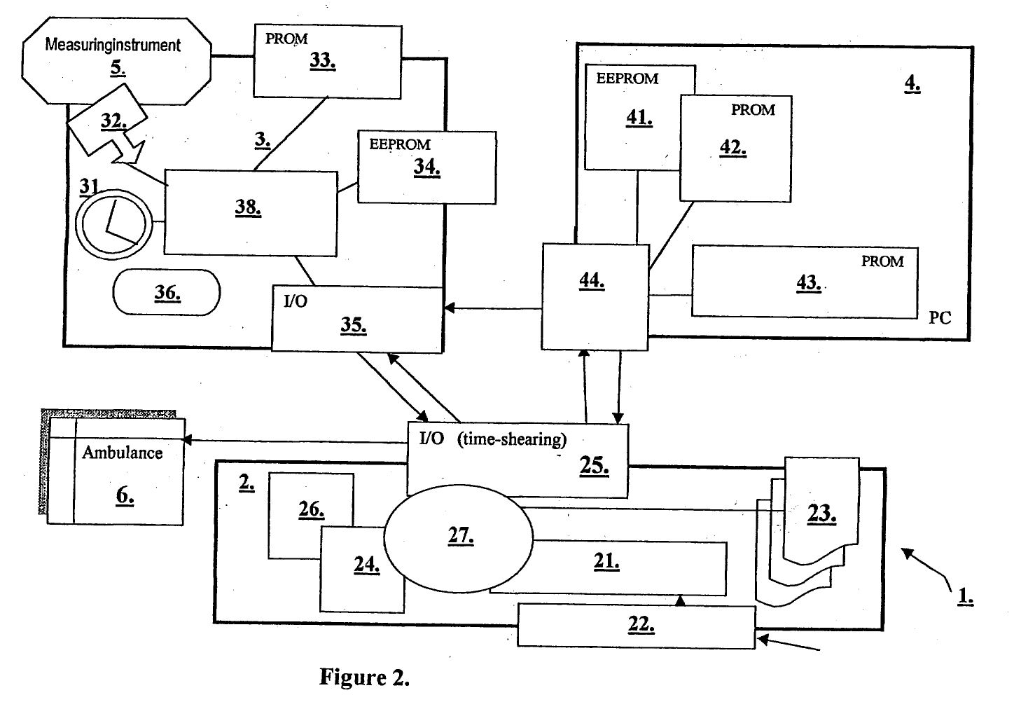

The system of apparatuses 1 comprises patient's units 3, physician's units 4 and one or more background databases. The patient's units 3 and the physician's units 4 are connected to the background database 2 (see FIG. 2). The background database 2 is equipped with an interface unit, which operates the connections with the patient's units and with the physician's units in “time sharing” system. The interface unit 25 of the background database 2 provides an emergency connection to the ambulance 6 or any other organization of quick medical help. The telemetric connection can be established by a telephone line, a radio frequency channel, using Internet or by any other way. It is allowed to use non-exclusively several different types of communications within the system of apparatuses 1. A coordination unit 27 performs receipt of calls and information incoming from the patient's units and physician's units, and forwarding them from the interface unit 25 to the data processing and evaluating unit 21. The said coordination unit 27 and the data processing and evaluating unit 27 can be preferably built up in the same micro-controller in the practice. A storage unit of medical data 24 built up on the base of a non-volatile memory, e.g. an EEPROM, which contains the identifiers of physicians participating in the system on one hand, as well as groups of patients belong to each physician and medical reports issued to patients on the other hand. A patient's data storage unit 26 is also connected to the coordination unit 27, the said storage unit contains comparative data, which are characteristic for each patient, observed and controlled by the system. A background storage 23 unit is also connected to the coordination unit 27, the said storage unit serves for systematized archiving of the collected data and for journalizing of events relating to operation of the system of apparatuses 1. The said background storage unit 23 contains also medical data at the expert's knowledge level and relations applicable for diagnosis of the group of diseases, i.e. those of hypertension in the present embodiment, for which the background database is built up (“evidence basic medicine”). A handling unit 22 is connected to the data processing and evaluating unit 21, the said handling unit enables data input, and it serves to the maintenance and updating of said evidence basic medicine stored in the background storage unit. The units of said background database 2 can be established by means of programming within a computer applicable for the said purpose, and they accomplish the functions described above. The patient's units 3 can either contain a measuring instrument 5, like a blood pressure meter, an ECG apparatus or combination thereof, or they can be connected to these instruments. The measuring instruments 5 are equipped with one or more sensors to detect the physiologic data and an output unit to forward then in form of electric signals. The patient's unit 3 is equipped with a signal handling unit 32, which is compatible with the measuring unit 5 and it filters, amplifies, smoothes and digitizes the signals incoming from measuring instrument 5 as required, and it forwards them to a data storage unit 34 based on an EEPROM by means of a control unit 38. The control unit 38 is the central one of the patient's unit 3, a program unit 33, a data storage unit 34 and a timer unit 31 are connected to it. The intelligence and equipment can be different, suitable to the task to be solved. The program unit 33 of patient unit 3 is preferably provided with the program, which controls the examinations according to the plan of examinations and control, i.e. with points of times of measurements, as well as with programs of processing and forwarding the measured data. These programs call up the background database 2 establish and maintain the connection with it and load the collected and partially processed and evaluated data via a background database interface unit 35. The background database interface unit 35 is a connecting unit, which transforms the data into signals to be transmitted by the applied information transmission channel. The patient's units 3 are preferably provided with a program, which controls the measurement of the actual physiologic data, e.g. for the measurement of blood pressure it controls the increase of pressure in the cuff, (for systolic pressure), then its decrease (for diastolic one) with simultaneous recording of the corresponding values of pressure. The patient's unit 3 is equipped with an input socket to connect the output plug of the measuring instrument with electric output in any case, when a separate measuring instrument is used for measurement. The patient's unit 3 can be equipped with a display unit 36, which warns the patient by a light or sound signal, either to perform the actual measurement or to the beginning of measurement, if an automatic instrument is used. This display unit 36 is not essential but an important part of the patient's unit. The background database interface unit 35 is connected to a telephone socket at usage of a wire transmission line is used; it can be connected e.g. to a mobile telephone preferably by means of an infrared connection for transmission by radio. The physician's units 4 are also parts of the system 1. A physician's unit 4 can be embodied as an intelligent terminal, an apparatus built into physician's PC or as a software product installed onto hard disk of the same PC which comprises the required logical drives. The physician's unit comprises an operating unit 42, a patient recording unit 41, a program storage unit 43 and an interface unit 44, which are connected to the operating unit. The patient recording unit 41 keeps record of addresses of patient's units 3 belonging to the physician's clientele, calling addresses of the background data base 2, furthermore the data and plans of examination and treatment of all patients, and data of as many recent measurements as required. The operating unit 42 contains programs applicable to change the plans of examination and those of therapy of patient's units 3. The program storage unit 43 contains programs suitable to perform search or query in the background database 2 or to download data therefrom. The interface unit 44 serves for establishing and maintaining telemetric connection and it complies with all required technical conditions therefor. The operating unit 42 maintains connection with the patient's units and the background database 2. The physician's unit 4 is equipped with all electronic units required to establish connection with the patient's units belonging to it, as well as to request, receive or send information, and to enable the physician to get knowledge and evaluate actual data and to change the treatment of patient according to his decisions.

The simplest embodiment of the system consists of a single physician's unit 4 and several patients' units 3. The background database 2 is within the physician's unit 4 and its function is essentially to reduce the physician's workload. The calls and receipt of information are performed automatically. The level of automatic processing and evaluation corresponds to the configuration of the physician's unit 4. Several physicians' units are served by a common background database. The service of a simple configuration comprises receipt of calls and selection of incoming data according to physicians' units 4. Services of higher level and complexity are provided by a background database according to its configuration.

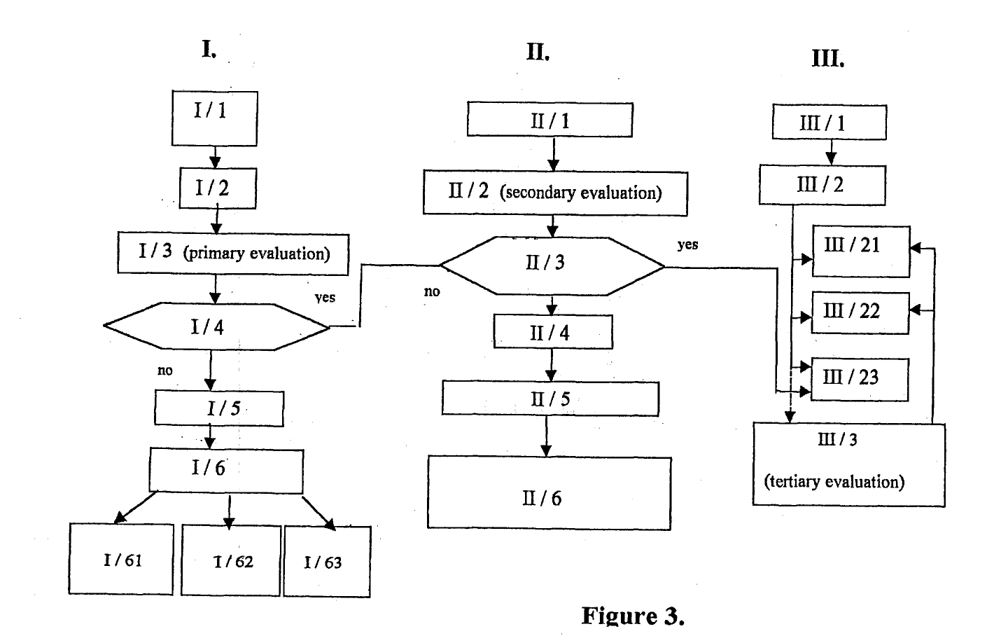

The operation of system of apparatuses 1 can be taken from the attached flowcharts (see FIGS. 3 to 5). The patients' units 3 perform physiological examinations of patients or warn patients to perform them in the prescribed points of time (see FIG. 3), then the units send data to the background database 2. The background database 2 serves to process and evaluate the data and connects the patients' units 3 with physicians' units 4.

In the FIG. 3 the used indicating numbers refer the below summarized proceeding steps.

I. PROCEDURE OF EXAMINATION

I/1 Check of points of time of examinations

I/2 Performance of examination

I/3 Primary evaluation

I/4 Alarm required?

I/5 Primary report

I/6 Receipt of an external call

I/61 Regular request of information from the background database

I/62 Issue of information for an occasional physician's call

I/63 Change of order of examinations or change of therapy

II. PROCEDURE OF DATA PROCESSING/EVALUATION

II/1 Regular information request from patient's unit

II/2 Evaluation of data (secondary evaluation)

II/3 Measure required?

II/4 Editing and sending a physician's report

II/5 Recording into journal and storage of data

II/6 RECEIPT OF CALLS: Extraordinary issue of information for physician's call; Measures for patient's call; Update of “Evidence Basic Medicine” and receipt of refreshing data are performed for development call.

III. PROCEDURE OF DIAGNOSIS/THERAPY

III/1 Receipt of physician's report

III/2 Measures taken after received physician's reports

III/21 Change of examination

III/22 Change of therapy

III/23 Alarm to ambulance

III/3 Usage of background information in the Evidence base; Tertiary evaluation

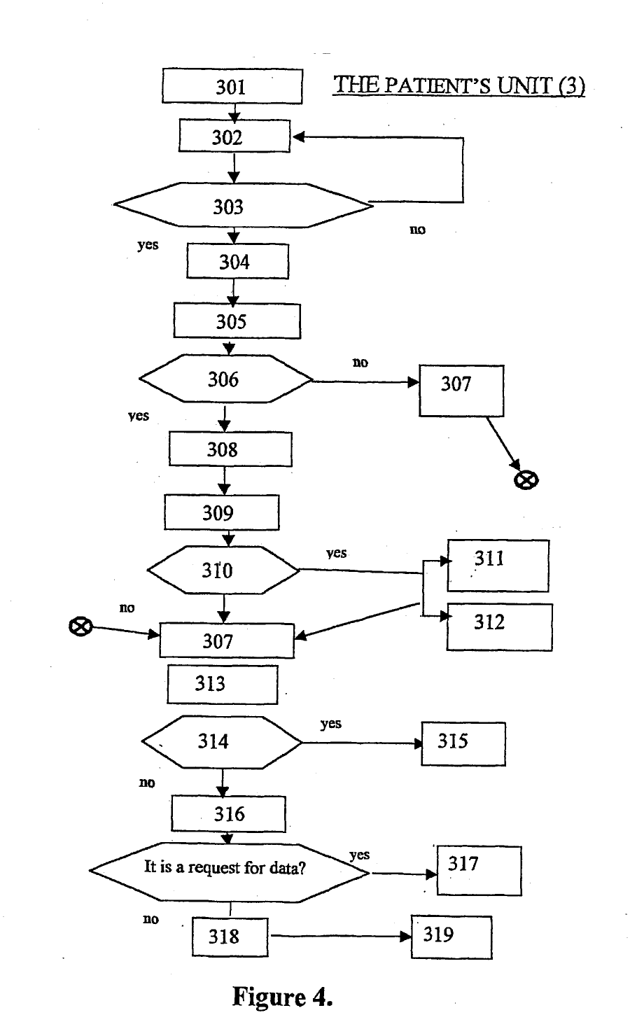

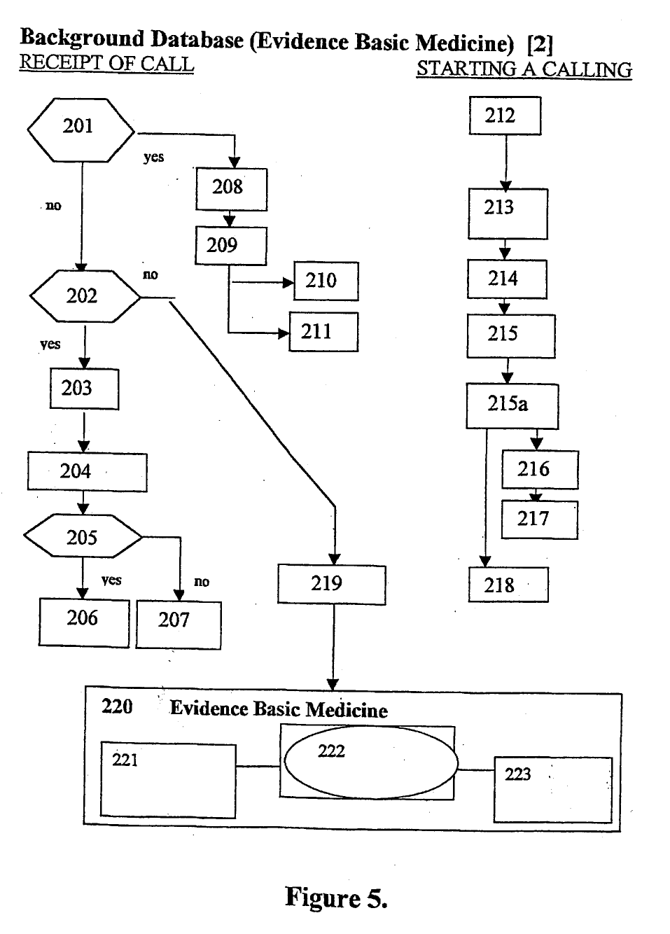

The details of operation of patients' units 3 are described with help of FIG. 4, those of the background database are described with help of FIG. 5. The control unit watches continuously the actual points of time 302 and compares 303 them with the time point of next measurement 301 called from the program of examination, which is stored in the program storage unit 34. When the programmed point of time is reached, a warning signal 304 appears on the display unit 36 that the patient should perform the measurement (e.g. of blood pressure), or informs the patient that the measurement has begun at the usage of an automatic measuring instrument. The control unit 38 calls the control program 305 of the blood pressure meter in case of measurement thereof from the program unit 33. The patient's reply signal 306 indicates that the measurement has started or it may be started automatically. At this point the patient's unit 3 performs the measurement according to the rhythm and within the ranges 308 prescribed. It receives measured 309 data by means of signal handling unit 32 during the measurement. If the patient's reply fails, the actual measurement cannot be performed, the control unit establishes a failure of patient's cooperation and stores it in the data storage unit 34. The control unit 38 analyses incoming data 309 and establishes as primary check 310 if any extraordinary measure should be taken. If the measured data require an urgent intervention in comparison to the programmed algorithm and/or to the data characterizing the normal status of patient, the control unit 38 issues a warning signal 311 (to repose, to go to fresh air, to take an extraordinary dose of medicament etc.) to the patient, at the same time it alarms 312 the competent physician, [by physician's unit 4] and the ambulance 6. The control unit 38 then stores the result of examination in the data storage unit 34 either as basic data or as compressed data using the program stored in program unit 33. The control unit 38 watches the points of time according to another program in the program unit, when it contacts with the background database and loads data from data storage unit 34 into background database 2 using the background database interface unit 35. The control unit analyzes calls 313 incoming to the background database interface unit 35 in another advantageous embodiment. If the call is a regular request for data 315 from the background database, the control unit loads data of measurements collected since the last request. If the call is a physician's one 316, and contains a request for data 317, the control unit loads data 317, otherwise, if the destination of call 318 is to change the order of examinations or the therapy, the control unit receives the changes of programs 319 and updates the corresponding parts of programs in the program unit 33 and in the data storage unit respectively. Any patient's unit 3 can be called from a physician's unit 4 and connected with it, which contains the calling code of said patient's unit. This fact means, that each physician can access exclusively the patients' units 3 of his own patients using his physician's unit 4. The access is secured by the own intelligence of patient's unit. A duplex contact exists between the patient's unit 3 and the physician's unit 4. The data in data storage unit 34 of patient's unit 3 can be loaded into physician's unit 4. The data of measurements called are readable but they cannot be overwritten or truncated. The physician can change the chronology of taking the medicaments and/or of control measurements. This program call be loaded immediately into patient's unit 3 replacing the previous one from the physician's unit 4. The patient's unit will issue warning signals 304 to the patient or control the measurements 305, 308 according to new or modified chronology. The background database 2 can call also extraordinarily the patient's unit 3 and request it for data. The data stored in data storage unit 34 are not erased in these cases but they remain up to the point of time of regular call for data. In the FIG. 4, (patient's unit [3]) the used indicating numbers refer the below summarized proceeding steps.

301 Time point of next measurement

302 “Real time”

303 Is it the time of measurement?

304 Warning to patient

305 Calling of measurement program

306 Is the patient's reply received?

307 Failure of compliance! To the time point “measurement failed” \mathring{x}

308 Performance of measurement

309 Processing of data

310 Is any intervention required?

311 Warning to patient

312 Background database; Information to physician, Alarm to ambulance

307 \mathring{x} Storage of data

313 incoming call

314 Is addressed to background database?

315 Regular data request, Sending of measured data

316 A physician's call

320 Is it a request for data?

317 Sending of data requested by physician's unit (4)

318 Change of order of examinations or of therapy

319 Receipt of program incoming from physician's unit

The procedures in the background database are connected with incoming calls 201 and messages 202 on one hand, and they are connected with calls 212 and messages 217 going out from the background database on the other hand. The calls are either regular ones 201 coming via interface unit 25 or from physician's units or extraordinary messages 202 coming from patients' units 3 into background database 2. If any message 203 comes from a patient's unit, the sending unit is identified first, then the message is forwarded to the data processing and evaluating unit 21 by the control unit 27. If the results of evaluation require an immediate intervention, the control unit 27 contacts with the actual physician's unit via patient's data storage unit 26 and sends an emergency report. The competent physician can immediately make contact with his patient on the base of said report, and he can alarm the ambulance, too, if needed. In any case if the connection between background database 2 and physician's unit 4 cannot be established, or the physician does not answer the alarm signal, or the only possible measure is to call the ambulance on the base of the evaluation, the coordination unit 27 sends alarm signal 206 to the ambulance together with all required data via interface unit 25. At the same time it sends verbal information to the patient's unit 3 to calm the patient or to request him to take some preventive measures. If no immediate intervention is necessary on the base of evaluation, the patient receives a message 207 about it via his patient's unit 3, the event is registered in the journal and the data are stored. If a physician's call 201 comes in, the physician's unit 4 should be identified 208 at first then the content of message is analyzed 209. If detailed data of a patient is requested, the data processing and evaluating unit 21 makes an extraordinary report and the control unit 27 sends it to the concerned physician's unit 4 via interface unit 25. The answer will be sent by similar way 211 if the call can be answered form the database of medical field. The physician is enabled by the system of devices 1 to make search or choice of information in the background storage unit 23 i.e. in the “evidence basic medicine” of the background database 2. The coordination unit 27 keeps record of points of times of regular requests of data from all patient's units 3, it connects to the actual patient's unit 3 via telemetric connection and calls 213 the results of examinations made since the last regular call. The results of examinations are processed in two complex connected steps, based on the characteristic data of patient 214 having the patient's unit 3 from the patients' database 26, the diagnosis 215 called from the database of special field of medicine in background storage unit 23 relating to anomalies resulted from evaluation of measured data and being as near as possible to them, and other algorithmic relationships. The results of analysis and evaluation are completed with detailed background information 216 and sent as regular medical report 217 to the concerned physician's unit 4. The events of sending reports 218 are entered into journal and the results are stored into background storage unit 23. If a call comes to the background database neither from a patient's unit 3, nor from a physician's unit 4, it originates from the working group of medical research and development and it is destined to update the database of “evidence basic medicine”. The update may be replacement of existing data, algorithms, standards and limit values with new ones, insertion of new information and extension of database. In the FIG. 5 (background database, i.e. evidence basic medicine [2]) used indicating numbers refer the below summarized proceeding steps.

200 receipt of call 200a initiation of a call

201 Is the physician calling?

202 Is the patient calling?

203 Identification of the patient's unit (3)

204 Analysis of the message

205 Is intervention required?

206 Message to physician; Message to ambulance; Reply to patient.

207 Reply to patient unit; Message registered data stored.

208 Identification of the physician's unit

209 Analysis of message: addressing for reply

210 To the data of a determined patient

211 To the Evidence Basic Medicine

212 Calling and identification of a patient unit (3)

213 Calling and storing of measured data

214 Analysis of data in comparison to the patients' own data

215 Analysis of data in comparison to the data of Evidence Basic Medicine

215a Analysis of data considering to knowledge including in Evidence Basic Medicine

216 Editing a physician's report

217 Sending a physician's report to the physician's unit concerned (4)

218 Registration into journal, storage of data

219 Check of rights

220 Evidence Basic Medicine

221 Replacement (refreshment) of old data

222 Recording and searching system

223 Extension with new data

The system of apparatuses according to invention embodies a practical configuration, which provides technically a completely new system of medical service and control. The main features and advantages of new system are as follows:

The patient is practically continuously under around the clock observation.

The observation oversteps the known methods of collection and registration of data. It controls not merely the observation of prescribed cure by the patient but also it controls its physiologic effect and continuously informs the physician about it.

There are, however solutions, which observe the lifestyle of patient and collect data about it. The evaluation of measured data by means of high-level algorithms is also known.

It has not existed a method, which evaluates the data simultaneously with processing and which provides interactive feedback, i.e. the physician's intervention.

Several telemetric systems are known, which establishes connection between the patient and his physician. No solution has been known up to now, which technically enables the physician that he could access an information database at the level of a conference of specialists simultaneously with connection with the patient. According to this solution the patient meets always his physician. The center of this system is in fact the background database 2, but it is a virtual unit for the patient, as he receives all the medical advices and prescriptions from his personal physician.

The attending physicians and those serving in National Health Service can treat a great number of patients observing their individual characteristics only by application of said technical solution according to invention. The solution takes off the load of observation and surveillance and of all-round analysis of data from the physician and enables him to utilize his knowledge on the base of carefully prepared results of examinations and to call complementary information from the background database 2 either relating to an individual patient or relating to up-to-date knowledge of medical literature connected to the assumed diagnosis.

The system according to invention enables financial economy. It reduces the number of out-patients being treated. Those, who should be nevertheless treated, they can begin either ambulant or in-bed treatment with a highly prepared collection of results of examinations. In the case of hypertension all patients can be separated, who suffer from “white cloak effect” only, as well as those having erroneously diagnosed cause of hypertension because of deficient data of examinations and receiving an inadequate treatment by this reason. The economy of health insurance system arises in the reduced number of patients to be treated, as well as in reduced number of visits and/or days of treatment in hospital, furthermore in the reduced number of measuring instruments to be used for examination of outpatients, too. The physician can apply less time to each patient, nevertheless, the monitoring of therapy and attendance are continuous. This results in considerable reduction of relative frequency of apoplexy, infarct and other grave consequences of hypertension, as well as the number of disabled persons, and costs of their provision are reduced by this way. The acquisition costs of physician's units can be considered as minimal in comparison with the costs of measuring instruments, which they replace. The patients by their own patient's units 3, the price thereof is quite not high in comparison with ones of widely used telecommunication and entertaining equipment household and do it yourself machinery being in fashion nowadays. The background databases 2 can be established and run effectively in an academic or clinical research and development center of a determined special field of medicine, because these institutions can perform their regular upkeep. The large quantity of on-line incoming data represents an inestimable value for the research and development on the other hand. The research center receives information about participating population of patients, which can be processed and evaluated by methods of statistics concerning its specific field of research, in exchange for its services.

The system of apparatuses according to invention enables the establishment and maintenance of this versatile, beneficial and advantageous system of connections.

1. A system of apparatuses (1) for examination of status and readiness for cooperation (compliance) of patients under treatment and/or for preparing their therapy, using remote data processing, the said system comprises patient's units at the persons being under observation and/or treatment and at least one unit installed at the physician and/or in a health center, characterized by, that
The patient's units (3) are equipped with:
An input connection, which is compatible with the output of an instrument (5) measuring one or more physiological data of the patient;
A signal handling unit (32), which filters from noise, amplifies and/or digitizes the incoming measured data according to necessity;
A control unit (38) equipped with a program unit (33), a data storage unit (34), a background database interface unit (35), a real time unit (31) and a display unit, issuing preferably a warning light or sound signal indicating the points of time to perform the actual examinations (36), which controls the said units;
A program unit (33) connected to the said control unit (38) and provided with a program, which controls the measurement of physiological data performed by the measuring instrument (5) among other functions;
A data storage unit (34) connected to the said control unit (38), which contains algorithms to process and evaluate the measured data and stores the measured data;
A background database interface unit (35) connected with the said control unit (38), which is applicable to establish connection with the background database (2) of the system of apparatuses to transfer data to or receive them from the said database;
Furthermore the physician's units (4) are equipped with:
An interface unit (44) designed for I/O operations to send immediately data to or receive them from a determined group of patient's units (3) and perform the same transfers with the background database (2) of the system;
Finally, the system of apparatuses (1) comprises one or more background databases (2), which are equipped with:
An interface unit (25) designed for I/O operations, which establishes and maintains simultaneous connections functioning in time-sharing mode with the patient's units (3) and the physician's units (4).

2. The system according to claim 1 characterized by that the physician's unit is equipped with an operative unit (42), which contains a system of programs applicable to change plans of examination and treatment in the patient's units (3) pertaining to it.

3. The system according to claims 1 or 2 characterized by that the background database (2) is equipped with a coordination unit (27), which provides sending of data to patient's units (3), as well as to physician's units (4), and sends alarm signal to ambulance of emergency intervention (6) if necessary, furthermore with a data processing and evaluating unit (21) connected to the said coordination unit (27), which processes and evaluates data incoming from the patient's units (3), draws up physician's report for physician and stores data for further usage in a background storage unit (23), using programs loaded into it.

4. The system of apparatuses according to claim 3 characterized by that the background database (2) is equipped with a background storage unit (23), which also contains data and algorithms of diagnoses and therapies (“evidence basic medicine”) pertaining to the special field of medicine of the background database (2) and with a data handling unit (22) connected to the said data processing and evaluating unit (21), which serves to insert data for refreshing the “evidence base medical” database.

5. The system of apparatuses according to any of claims 3, 4 characterized by that the background database (2) is equipped with a patients' database (26) containing the identifiers and data characterizing the status of patients, and a physician's database (24) containing clienteles pertaining to each physician, and both databases are connected to and controlled by the said coordination unit (27).

6. The system of apparatuses according to any of claims 3-5, characterized by that the said background unit (23) contains a journal to record events arisen in the system, the measures taken and other data.

7. The system of apparatuses according to any of claims 3-6, characterized by that the background database or preferably its background storage unit (23) contains the evaluating algorithms of mathematical statistics applied to medical purposes.

8. The system of apparatuses according to any of claims 1-7, characterized by that the physician's unit (4) is equipped with a program storage unit (43), which is provided with programs to perform searches and queries in the background database (2).

9. The system of apparatuses according to any of claims 1-8, characterized by that it is equipped with a patient recording unit (41), which contains plans of examinations and treatment of the patient's units pertaining to it, the data of patients, as well as data of the required number of recent medical reports, recalled from one or more background databases (2).

10. The system according to any of claims 1-9, characterized by that the results of examinations and their processed data cannot be changed or erased by the physician's unit (4).
